(19) United States
(12) Patent Application Publication
(10) Pub. No.: US 2012/0224994 A1
(43) Pub. Date: Sep. 6, 2012

(54) AIR CLEANING APPARATUS

(76) Inventor: John William Steiner, Nundah (AU)

(21) Appl. No.: 13/322,342

(22) PCT Filed: Feb. 24, 2010
(86) PCT No.: PCT/AU2010/000211

§ 371 Date: Jan. 25, 2012

(30) Foreign Application Priority Data
Feb. 24, 2009 (AU) 2009900787

(51) Int. Cl. A61L 9/03; A61L 9/02

(52) U.S. Cl. 422/3; 422/105; 422/111

(57) ABSTRACT

An air cleaning apparatus for deodorising or cleaning of air containing noxious or toxic odours is disclosed. The apparatus includes a controller that is a PLC, an air pump, a solenoid, a manifold, a pressure gauge, an air supply line and air supply lines in fluid communication with the manifold. It also includes a first tank that contains a deodorising solution, a second tank contains a detoxifying solution and the third tank contains an oxidising/disinfecting solution. These are the components that are delivered into the air to deodorise and/or clean the air. The apparatus further includes outlet conduits or lines which are in fluid communication with in line filters which as shown are in fluid communication with solenoids. The apparatus further includes supply flow conduits which are in flow communication with an evaporation assembly where a phase change of said decontamination solution occurs to convert it into an activated molecular species.

### BACKGROUND

1. Field of the Invention

The present disclosure relates generally to air cleaning apparatus that converts specific chemical solutions into transitional/molecular vapours which can be used to reduce or eliminate noxious odours and/or toxic gases emitted from a wet well of a sewage pumping station or other confined air spaces, such as sewage treatment plant, garbage chutes or working environments.

The disclosure specifically describes air cleaning apparatus for the chemical dissociation of disagreeable odours and toxic gases present in air streams escaping from sewage systems and/or removal of gaseous contaminants from air in a confined space. Specific compounds present in these air streams contain sulphur, such as hydrogen sulphide, mercaptans and others, as well as nitrogen, carbon and hydrogen containing compounds, such as skatole, indole and others.

2. Discussion of the Background Art

Over the years many different methods for the removal or elimination of these types of contaminants have been utilised. These include but are not limited to the deployment of commercially available biofiltration, activated carbon filtration, adsorption/absorption and chemical conversion filters, ozone treatment, dosing with gaseous compounds, such as chlorine, adding fragrances, using oxidising, reducing, and/or neutralising agents and/or vapours of essential oils, using ultraviolet irradiation, using masking agents, converting pre-designed solutions into aerosol or fine mists, electro trapping, incineration and dispersion among others. All of these techniques are stand-alone systems and are neither integrated, nor are they sequentially synchronised with some of the bioactivities of the sewage system for example, which is largely responsible for initially creating the problems. Many of these methods work under certain conditions but they lack automation, operational flexibility and fail to accommodate regular changes of the diurnal/hydrodynamic activities within the sewage system which alters significantly within a 24 hour operating phase. Apart from the disclosure described herein, a single method for treating the above-described problem with regards to reliability, cost effectiveness and consistency has not yet emerged.

The above-mentioned methods do not target the fundamental causes of the malodour and microbial contamination. The air cleaning apparatus of the disclosure is a direct method addressing this problem which embraces several concepts of microbiology, chemistry, robotics, management of information technology and analytical instrumentation. There is not at present a single source that addresses the complexity of this topic in a thorough fashion. Specific knowledge of bioactivity of the biomass, volume and detention time within the overall waste handling system has not yet received true consideration. Particularly reference is made to three bio-waste handling systems, such as the sewer, composting and garbage chutes in high rise buildings. It would seem from observations made hereinafter by the inventor and as illustrated in FIG. 6 that diurnal/hydrodynamic activities within the main sewer line clearly suggests the there are four well-defined cycles, which also correlate to four main malodour/toxic gases emission cycles. These cycles are largely influenced by repetitive and highly regular behaviour patterns of humans. Thus for example it is the habit of humans after getting up in the morning to proceed immediately to the toilet. This highly synchronised action, early in the morning, leads immediately to a nutrient overload of the sewage systems in highly populated geographic regions within Australia.

The dynamics of malodours from garbage chutes is also influenced by the relationship of microbial activities, composition and volume of biomass, temperature, relative humidity and detention times. As the bin collecting rubbish from the chute becomes fuller, the rate of malodour emission increases. This cycle is stopped when the bin is emptied. However, due to economic considerations bins may be emptied once a week.

The main problems with existing methods are that the abovementioned cycles are not taken into account in reduction or elimination of noxious or toxic gases being emitted from air streams. Thus for example, conventional treatments are neither synchronised, nor integrated with the daily operational factors of the waste handling system, such as mass/volume, microbial activities and detention times.

A most practical and proven application of this disclosure is its application along the South East coastline of Australia. In this location there is an extremely high growth in population but there is no corresponding expansion of the capacity of the mains and gravity feed lines delivering raw sewage to treatment plants. Overloading the mains and gravity feed lines leads to the emission of malodours, toxic gases and air-borne microbes such as bacteria and viruses into escaping air streams. Connecting the air cleaning apparatus of the disclosure to sewerage treatment plants or to wet wells of pumping stations or vent pipes reduces the release of these contaminants and provides extra capacity for mains and gravity feed lines. Physical expansion of the mains would generate a huge expenditure for ratepayers, which may counteract the current housing investment trend and, as a consequence, reduce the population density along the South East coastline of Australia.

In summary, the air cleaning apparatus of the disclosure is offering a new technology with an innovative conversion of specifically designed chemical solutions as hereinafter described, utilising a specifically designed procedure to convert the chemical solutions into transitional/molecular vapours and subsequently deliver these vapours via a specific pathway to the source of the odour and/or toxicity, to counteract the problems described above as well as associated public health problems, toxicity problems and public nuisance problems. The sources of these problems are related to toxic gases, malodours, and viral and/or microbial contamination at domestic, commercial and industrial sites. The chemically active compounds can be drawn from decontamination chemical solutions including oxidising, detoxifying, deodorising, sterilising, antiseptic, antibacterial, antimicrobial and antiviral compounds. After conversion air, and not a solvent, is used to deliver the active chemical constituents to the contaminated site. Another significant distinction of this disclosure, when compared with others, is the ability to convert a solution into transitional/molecular vapours and not into a fine mist or aerosol. This air cleaning apparatus of the disclosure is capable of performing sequentially and/or concurrently delivery of the abovementioned decontamination chemical solution to reduce or eliminate the abovementioned problems of the prior art.

### SUMMARY

The air cleaning apparatus of the disclosure includes:

(i) a controller for receiving real-time data from at least one sensor to control the operation of the air cleaning apparatus; and

(ii) at least one tank containing a decontamination solution;

(iii) a source of compressed air in fluid communication with the said at least one tank; and

(iv) an evaporation housing having at least one coil assembly in flow communication with the said at least one tank wherein said evaporation housing has an air inlet and an air outlet wherein the air outlet in use is in fluid communication with a wet well source of noxious or toxic odours whereby during operation of the air cleaning apparatus said at least one decontamination solution is delivered under pressure to the said at least one coil assembly under the influence of the controller wherein a phase change of said decontamination solution occurs into an activated molecular species which is transferred into the wet well by air passing through the air inlet and the air outlet to reduce or eliminate said noxious or toxic odours from the wet well.

The controller may be one or more of an electronic controller. The controller may also be a programmable logic controller (PLC controller) and an integrated chip.

Instead of an evaporation housing other subsystems for achieving the same result may be used.

The decontamination solution for use in the disclosure is useful in causing a chemical dissociation or converting compound(s) which are the cause of the noxious or toxic odours into non-toxic or non-malodorous compounds which are harmless and not offensive to the olfactory receptors. It will also be appreciated herein that the term “noxious or toxic odours” as used herein also includes within its scope compounds including gases as well as micro-organisms such as air borne viruses or bacteria.

More preferably there are provided a pair of tanks and most preferably there are provided at least three tanks wherein a first tank contains a deodorising solution (i.e. Group A) a second tank contains a detoxifying solution (i.e. Group B) and a third tank contains an oxidising/disinfecting solution (i.e. Group C).

Examples of Group A solutions are one or more of ethanol, aldehydes, ketones, chloroxylenol, benzalkonium chloride, essential oils, cyclodextrins, isopropyl alcohol, orris concrete, oleoresins, orris root, chlorohexidine and esters. Examples of Group B solutions are organic solutions including di-isopropyl amine, diglycolamine or aminoethoxy ethanol, monoethanolamine, pyrimethamine, acetonitrile, vinorelbine ditartrate, methonal, xylitol and n-hydroxyethyl piperidine as well as inorganic solutions including ammonium chloride, ferric chloride, ferric hydroxide, caustic soda, hydrogen peroxide and others. These solutions may be used alone or in combination.

Examples of Group C solutions include one or more of ethanol, isopropyl alcohol, water, acetic acid and hydrogen peroxide.

Preferably the Group A, Groups B and Group C solutions are delivered in sequential order to the evaporation housing and in this particular embodiment the evaporation housing may have at least three coil assemblies having a first coil assembly which is in fluid communication with the first tank, a second coil assembly which is in fluid communication with the second tank and a third coil assembly which is in fluid communication with the third tank.

The evaporation housing may also have at least one or a plurality of fans, air blowers or compressors adjacent to the air inlet so as cause a forced draught of air to flow from inlet to outlet to transfer the activated molecular species of Group A, Group B and Group C solutions into the wet well source of noxious or toxic odours.

The evaporation housing in a preferred embodiment allows water to be delivered to fluid injectors which form part of each coil assembly so that an adjacent coil assembly may be cooled after each coil assembly is turned off by the electronic controller. Preferably this procedure may be operated by the electronic controller or a PLC in conjunction with an array of integrated chips.

The proportions of the main active compounds present in Group A can be in the ratio of 1-50% to 50-1% by weight of the solution. Group B is preferably undiluted and Group C can be mixed in any suitable proportion to achieve the desired result and can be from 1-21% by weight of the solution.

Using a real time controller measurement and control mechanisms combined with multiple sensors allows predetermining the required dosing rates, which then determines whether a light or heavy treatment and how many treatments are required, before any treatment is administered. The real time option combined with advanced automation and using multiple sensors, adds a new dimension in the operational flexibility of this air cleaning apparatus of the disclosure when compared with conventional devices and methods offered in this field. The technological superiority of this disclosure significantly reduces operation and maintenance costs and, in addition, provides a much higher efficiency, reliability, convenience and effectiveness than other devices and methods offered by the prior art. The dosing rate and number of treatments are determined electronically and minute amounts (e.g. 0.8 up to 6.0 millilitres) of solutions can be converted into transitional/molecular vapours per treatment. The chemical agents consisting of Groups A-C can be injected, at a controlled rate, into the abovementioned fluid injectors which are associated with each coil assembly in the evaporation housing which are then converted in a sequential order into transitional/molecular vapour and thereafter instantly delivered, using air as a carrier, into the wet well source of noxious or toxic odours which can be any confined air space.

Different real time operational and analytical data are regularly compared with present threshold values, which ensure that the air cleaning apparatus of the disclosure operates continuously in an optimised manner. Combining the highly flexible operating conditions of this apparatus, the properties and potency of the solutions combined with the appropriate dosing rate and number of treatments, make it possible to eliminate undesirable components, such as airborne viruses, odorous and toxic/gaseous compounds and micro-organisms, from contaminated air streams which are present in many different environments, such as wet wells, airports, office buildings, hotels, aeroplanes, transit terminals and garbage chutes.

It is possible using the air cleaning apparatus of the disclosure that a very simple apparatus can be used which only requires an electronic controller such as a PLC, and a single coil assembly located in the evaporation housing which is in flow communication with a single tank containing a decontamination solution. There may also preferably be only a single fan or air blower adjacent the inlet of the evaporation housing together with an air pump providing one example of a compressed air source. There may also be included in this embodiment a single flow conduit between the air pump and the single tank and a single flow conduit between the single tank and the single coil assembly which also has a flow control valve and a solenoid.

An air cleaning apparatus in accordance with this disclosure may manifest itself in a variety of forms. It will be convenient to hereinafter describe at least one embodiment of the disclosure in detail with reference to the accompanying drawings. The purpose of providing this detailed description is to instruct persons having an interest in the subject matter of the disclosure how to carry the disclosure into practical effect. However it is to be clearly understood that the specific nature of this detailed description does not supersede the generality of the preceding broad description. In the drawings:

### BRIEF DESCRIPTION OF THE DRAWINGS

FIG. 1 shows a schematic flow sheet of the process of the disclosure;

FIG. 2 is a detailed view of a liquid/gas separator used in the process shown in FIG. 1;

FIG. 3 is a detailed view of a noxious or toxic odour detention apparatus used in the process shown in FIG. 1;

FIG. 4 is a detailed view of an evaporation assembly used in the process shown in FIG. 1;

FIG. 5 shows the relationship of time versus volume of solutions detailed to the injectors shown in FIG. 4; and

FIG. 6 shows a correlation of dosing and treatment rates, noxious odours emission rate and/diurnal hydrodynamic activities.

### DETAILED DESCRIPTION OF THE PREFERRED EMBODIMENT

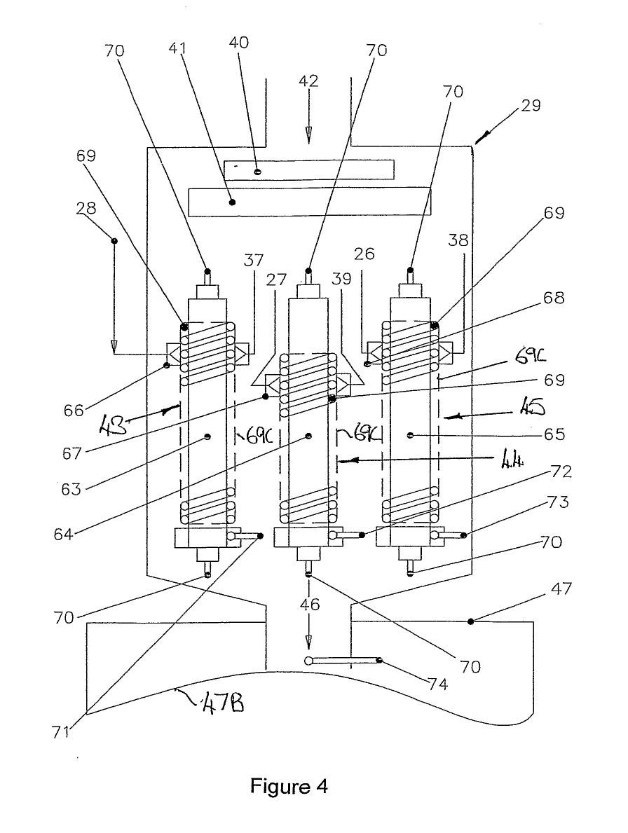

In addition there is also provided Table 1 which shows an integral part of using real time thermometric data to ascertain heating rates, temperature profiles of evaporation coil assemblies shown in FIG. 4 and deployment of the data for diagnostic purposes for monitoring performance of the air cleaning apparatus of the disclosure and to utilise the data combined with the PLC to run an automated safety interlock system.

Also there is provided Table 2 which sets out the threshold settings in order to optimise operating parameters of the air cleaning apparatus shown in Table 1.

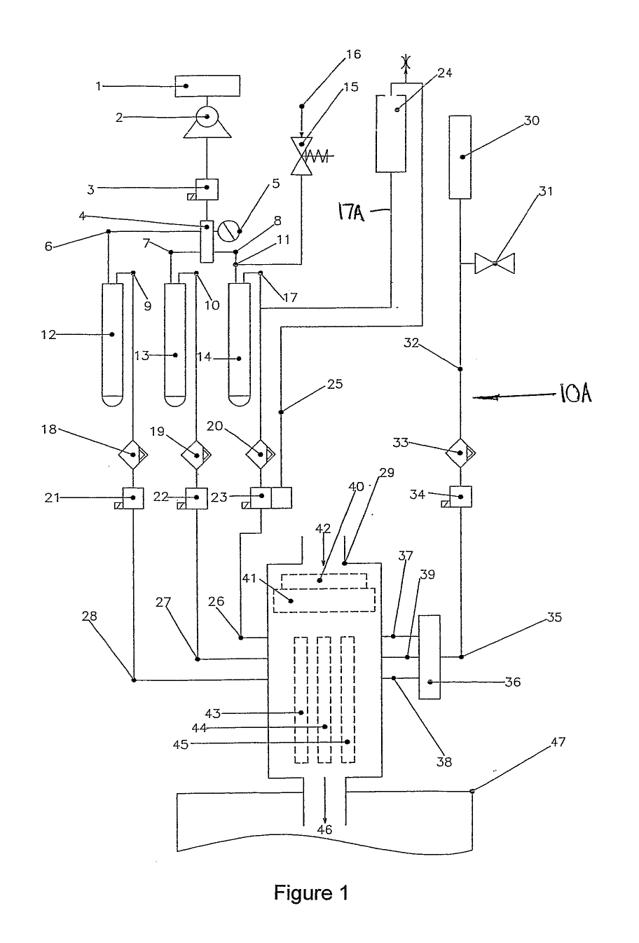

In FIG. 1 there is shown air cleaning apparatus 10A which includes PLC 1, air pump 2, solenoid 3, manifold 4, pressure gauge 5, air supply line 6 and air supply lines 7 and 8 in fluid communication with manifold 4, tanks 12, 13 and 14 containing treatment or decontamination chemicals, outlet conduits or lines 9, 10 and 17 which are in fluid communication with in line filters 18, 19 and 20 which as shown are in fluid communication with solenoids 21, 22 and 23. There are also provided supply flow conduits 26, 27 and 28 which are in flow communication with evaporation assembly 29.

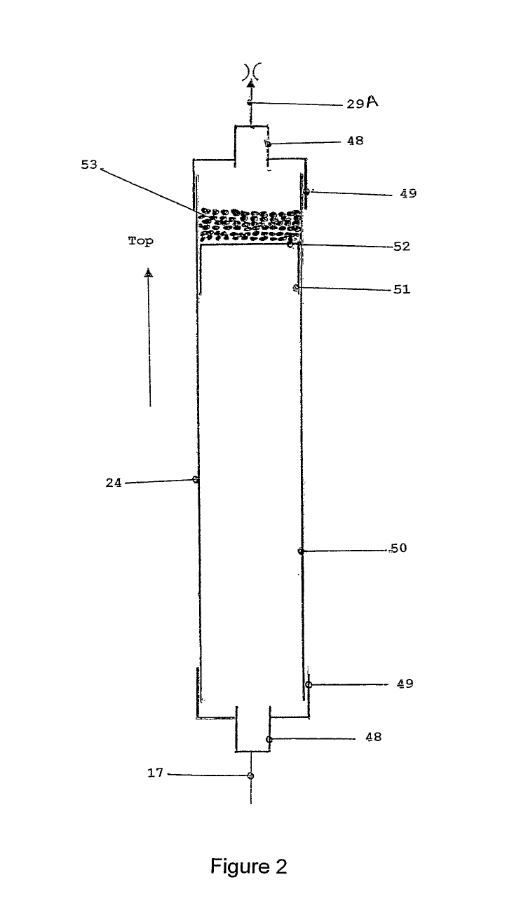

In relation to outlet conduit 17 which contains liquid and gas which needs to be separated before introduction into solenoid 23 there is a bypass line 17A which is in fluid communication with a liquid/gas separator 24. This procedure is more fully described in FIG. 2.

In evaporation assembly 29 there is shown air supply inlet 42 and adjacent fans 40 and 41 which can operate at different speeds. There is also provided air outlet 46 and evaporation coil assemblies 43, 44 and 45.

There is also provided town water supply connection 30, supply conduit 32, flow control valve 31, in line filter 33, solenoid 34 and water supply conduit 35 which passes through manifold 36 and into three separate supply conduits 37, 38 and 39 which are in flow communication with coil assemblies 43, 44 and 45 as shown in FIG. 4.

FIG. 2 is a detailed view of the liquid/gas separator 24 and includes supply line 17A. There is also shown a seal 49 which interconnects inlet member or fitting 48A and PVC pipe 50. There is also provided sleeve 51 and sieve 52. Sleeve 51 snugly fits inside pipe 50 and placed on top of sieve 52 are titanium balls 53 to break up tiny little air bubbles as hereinafter described. Supply line 17A is connected to inlet 48A of fitting 49.

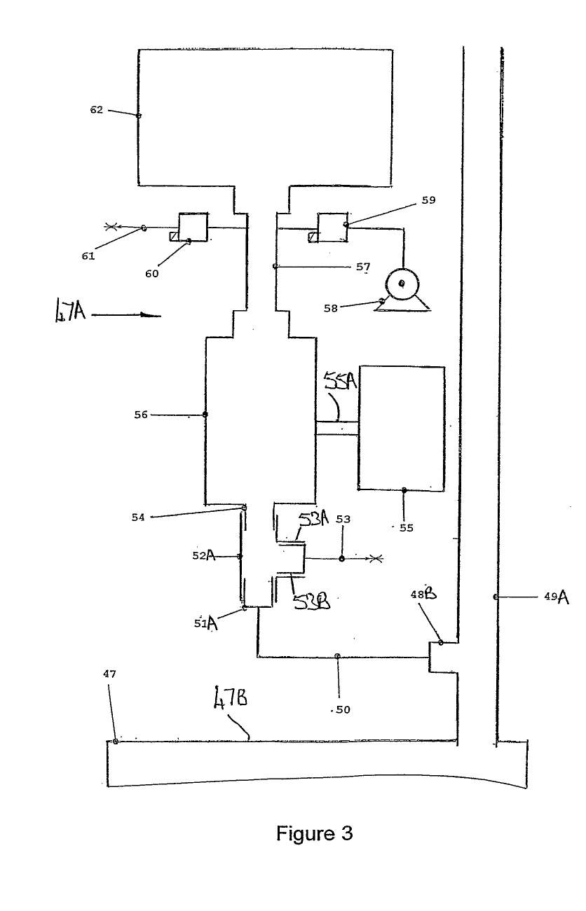

In FIG. 3 there is shown detection apparatus 47A which is connected to vent pipe 49A of wet well 47 shown in FIG. 1. Detection apparatus 47A is a continuation of FIG. 1 along wavy line 47B.

In FIG. 3 there is shown vent pipe 49A and connector 48B interconnecting vent pipe 49A with gas conduit 50A. There is also provided T-junction 52A having bypass passage 53A which has fitting 53B located adjacent thereto. Fitting 53B is in fluid communication with bleed passage 53. There is also provided fitting 51A which connects tube 52A with gas conduit 50A. There is also provided an airgate 56 having access port 54 which is connected to tube 52A. There is also provided step-in electronic motor 55 which drives air gate 56 through connector 55A.

There is also shown in FIG. 3 adaptor or connector 57 which interconnects air gate 56 to H₂S sensor 62. There are also provided bleed line 61, solenoids 59 and 60 and air pump 58.

In FIG. 4 there is shown a detailed view of evaporation assembly 29 which in addition to what is described in FIG. 1 includes coil supports or rods 63, 64 and 65, electrical terminals 70, injectors 66, 67, and 68, coils 69, and thermocouples 71, 72, 73 and 74. The coil supports or rods 63, 64 and 65 are the internal components of coil assemblies 43, 44 and 45 as shown in FIG. 1. There is also provided evaporation platform 69C.

In operation of the process of the disclosure as shown in FIGS. 1-4, contaminated air or gas accumulates or gathers in wet well 47 which is a container or reservoir of such contaminated air or gas or fluids having a gas/liquid interface or liquid slurries. This contaminated fluid flows into wet well 47 through an open top (not shown) by gravity and thus corresponds to a sump for example. Such wet wells 47 can be connected to each other by a reticulated network of pipes or conduits interconnected by pumping stations. Such network is in fluid communication with a sewage pumping stations connected via pipes to sewage treatment plants.

The contaminated air or gas in wet well 47 may be treated on the basis of a function of time combined with the threshold settings or using real time and measuring concentration of H₂S in wet well 47 and, at the same time, measuring meteorlogical and operating conditions of the air cleaning apparatus 10A and comparing analytical data with the values of the threshold settings. Using the air cleaning apparatus 10A in the “real time mode” the concentration of the H₂S in wet well 47 is measured with air gate 56 being open then within a delay of 60 seconds, the concentration value of H₂S is sent to the PLC. If the measured value exceeds a predetermined low value (see threshold settings in Table 2) then a light treatment will be administered, which practically means that low dosing and minimal treatment rates will be administered. To facilitate this treatment regime, the PLC sends signals to solenoids 21, 22 and 23 sequentially programmed by the PLC which determines a time delay between signals being sent to solenoids 21, 22 and 23. This means that chemical agent A, B and C from tanks 12, 13 and 14 are delivered to injectors 66, 67 and 68 as well as an external surface of each coil support 63, 64 and 65 and also coils 69 thereby creating evaporation platforms 69C as shown in phantom in FIG. 4. This provides a large surface area which results in a phase change of each chemical agent to an activated molecular species which provides that each chemical agent is in a gaseous and very active form. The molecular species of each chemical agent is then transferred into wet well 47 by air passing through entrance 42 and exit 46 of evaporation assembly 29.

Referring to FIG. 1, it can be envisaged that PLC 1 energises or de-energises air pumps 2 and 58, solenoids 21, 22, 23, 34 and 59 and 60 whenever required. It also energises or de-energises motor 55 that drives air gate 56, fans 40 and 41 and evaporation coil assemblies 43, 44 and 45. The solenoids 21 to 23 allow solutions stored in tanks 12 to 14 to be delivered to injectors 66 to 68. The solenoid 34 allows delivery of water to injectors 66 to 68. Before delivery of solutions or water, each evaporation coil 69 heats up. When an injector combined with an associated evaporation platform 69C reaches a temperature that is equal to the boiling point of the solutions (Group A, Group B and Group C), an injection is made. Thereafter each injector 66, 67 or 68 distributes the delivered solutions or water over the evaporation platform 69C designated for each injector. Due to the broad temperature profile of each coil 69, which has its hottest heating zone at the bottom of the coil, the solutions or water evaporate on the evaporation platform 69C as they travel slowly downwards towards the bottom hot heating zone. The vapours leaving the platforms are immediately taken away with fans 40 to 41 via duct 46 into the wet well 47.

An important part of the fluid mechanics underlying the disclosure is air pump 2. The primary function of the air pump 2 is to pressurise tank 12 (storing chemical agents Group A), tank 13 (storing chemical agent Group B) and tank 14 (storing chemical agents Group C). When air pump 2 is energised, then solenoid valve 3 also opens and air enters via manifold 4 to pressure gauge 5 and air supply lines 6 to 8 which are connected to tanks 12 to 14. After pressurisation of the tanks for 25 seconds, air pump 2 stops. If pressure in the tanks 12 to 14 is above 3.5 psi, high precision pressure relief valve 15 opens and closes when the pressure has decreased to 3.2 psi. The excess air is released through port 16. When tanks 12 to 13 are pressurised, the solutions will travel via supply lines 9 to 10 from the tanks through filters 18 and 19 and then to solenoid valves 21 to 22. If these solenoid valves are open then the solutions will flow via flow conduits or capillary tubing 27 to 28 to injectors 66 and 67. However, when tank 14 is pressurised, the air in tank 14 will diffuse through the hydrogen peroxide solution if present and thereafter escape as very tiny little air bubbles, which become visible in supply line 17. To remove the tiny air bubbles in the hydrogen peroxide solution, a liquid/gas separator 24 was developed and installed.

An integral part of this separator is illustrated in FIG. 2. It represents separation column 50 that is installed on a wall 3.6 metres above ground. For a tank pressure of 3.1 psi a head pressure of 2.2 metres is produced. The air bubbles combined with small amounts of solution via line 17A continuously emerge at the lower end of the column or pipe 50 but some continue their journey towards sleeve or fitting 51 which as stated above has nylon sieve 52 and titanium balls 53 to break up the air bubbles. Thereafter, the air bubbles escape through bleed pipe 29A and the hydrogen peroxide solution returns to line 17A.

FIG. 1 shows that line 17 is connected to filter 20 and solenoid 23. This solenoid has three ports as shown, which facilitates that one port opens when the opposite port closes. For example, when PLC 1 energises solenoid 23 then the port to solution supply line 17 opens and also the port to capillary line 26 connected to injector 68 opens but, at the same time, the port to bleed line 25 closes. When the power to this solenoid is turned off bleed line 25 opens and ports to line 17 and capillary line 26 closes. Bleed line 25 is connected to bleed passage referred to as 24A and 29A.

When solenoid 34 is opened, water connected to the reticulated water supply 30 flows through regulator 31, which is connected via a supply line 32 and filter 33. From there it then flows into the solenoid 34, which in turn, is connected with line 35 to manifold 36. This manifold consists of three additional ports: Capillary tubing 37 is connected with manifold 36 and injector 66. Capillary tubing 39 is connected with manifold 36 and injector 67. Capillary tubing 38 is connected to manifold 36 and to injector 68. This is shown in FIG. 4. Capillary tubing or flow conduits 37, 38 and 39 provide for injecting water from injectors 66, 67 and 68 into the evaporation platforms 69C This provides for cleaning of each platform 69C after use, dilution of the volume of water injected on each platform 69C and increase of boiling point and accelerated cooling of platforms 69C.

FIG. 4 also shows the location of thermocouples 70 to 73. These thermocouples facilitate monitoring and optimisation of performance and also allow real time monitoring of the heating/cooling profiles of evaporation coil assemblies 43 to 45, fan function of fans 40 and 41, and working of injectors 66, 67 and 68. In addition, the temperature measurement provides the necessary data to continuously run a temperature based diagnostic and auto safety interlock system that becomes active and continuously monitors the thermometric performance of the device before, during and after a chemical treatment is executed.

The symbols (M1 to M4) are used for convenience to understand the working of the thermometric monitoring system of the disclosure. M1 means temperature measurement 1. M2 means temperature measurement 2, etc. M1 H means temperature measurement of the upper temperature range whereas M1 L means taking a measurement of temperature in the lower range. M2 H means that M2 H is higher than M1 H but is lower than M2 HH. To explain this thermometric monitoring system of the device further, examples are given below.

M1 H and M1 L provide a means of measuring ambient temperature in the evaporation assembly 29 before commencement of a chemical treatment. (Seasonal changes will require altering the settings of temperatures threshold for M1 H and M1 L).

M2 H and M2 HH signify that M2 H shows the temperatures of heating evaporation coil assemblies 43 to 45 and when M2 H is reached, the electrical power to the coils is turned off. [The deodorising and detoxifying solutions (Group A and Group B) are injected before M2 H is determined]. Due to the thermal mass of evaporation coil assemblies 43 to 45 and evaporation platforms 69C the temperature will still rise until it peaks and, at that point, the value of M2 HH is determined. Thereafter, the heating profile reverses into a cooling profile and the cooling phase proceeds in full progress.

M3 is determined before injection of the oxidising/disinfecting solution (Group C).

M4 is determined at the point the cooling cycle is complete. If the measured temperature is higher than the stored threshold temperature (M1 L, M1 H, M2 H, M2 HH, M3, M4), an error message will appear and the PLC 1 will discontinue with the operational run.

TABLE 1 shows the thermometric manipulation of data to assist further with the interpretation and differentiation of M1 L, M1 H, M2 H and M2 HH, M3 and M4. TABLE 1 makes specific reference to the following examples. M1 L and M1 H are determined before commencement of heating the coils 43 to 45 and evaporation platforms 69C. If the defined temperature range is outside the predetermined threshold, the operating cycle is terminated. M2 H is determined when the power to the coils are switched off but, due to the thermal mass of the coils and evaporation platforms 69C, the temperature still rises even without power. M2 HH is determined when the temperature of the coils reaches a plateau after which the coils and the evaporation platforms 69C commence the cooling cycle. M3 is determined before injecting the oxidising/disinfecting solution. M4 is determined upon finishing the cooling cycle.

FIG. 4 also shows coil supports 63 to 65 to ensure that thermocouples 71 to 73 have a direct connection with evaporation coil assemblies 43 to 45. (The thermocouples 71 to 73 monitor the temperature of evaporation coil assemblies 43 to 45). At regular intervals temperature values provided by thermocouples 71 to 73 are sent to the PLC 1 which interrogates the data, makes a comparison with the threshold values and decides which task needs to be done next. During the heating, cooling and before the resting cycle, the temperature is measured at regular intervals (M1 to M4) and produces a temperature profile of the evaporation coil assemblies 43 to 45. Each coil assembly 43 to 45 provide a contact 70 to connect to electrical power (240V AC).

Each treatment is comprised of the following cycles: heating/evaporation cycle, cooling cycle and resting cycle. The three cycles are equivalent to one treatment cycle. Heating cycle means that each evaporation coil assemblies 43 to 45 is energised for 25 seconds. This energising cycle provides a heating rate of approximately 7 C°/s to each of coils 63 to 65 and evaporation platforms 69C. As the temperature gradient of the coils is rather wide, the heating rate (7° C.) can only be achieved over a small area of each coil. At the beginning of the heating cycle, two chemical agents (Group A and B) are injected onto the evaporation platforms 69C which transports the solutions to two separate coil assemblies 43 and 44. The third chemical agent (Group C) is injected onto the third coil assembly 45 and evaporation platform 69C during the cooling cycle. After the energy supply to each coil is terminated, the cooling cycle commences.

The cooling cycle means the cooling of the coil assemblies and evaporation platforms 69C, which are accelerated with fan 41 which turns on when the evaporation coil assemblies 43 to 45 are turned off. The appropriate time required of cooling each coil assembly to ambient temperature takes approximately 4 minutes. However, if solenoid 34 is activated, then water 30 is delivered to the evaporation coil assemblies 43 to 45, which accelerate the cooling cycle. Adding water to coil assemblies 43 to 45 during the cooling cycle provides the extra benefit that the coils remain clean. The resting cycle means that the cooling cycle is completed and the resting cycle may run for 125 seconds. If the treatment frequency needs to be increased, then the resting cycle needs to be decreased.

Another unique feature of the disclosure is the evaporation assembly 29, which is shown in more detail in FIG. 4. The assembly consists of a stainless tube that houses evaporation coil assemblies 43 to 45, evaporation platforms 69C, fans 40 to 41, injectors 66 to 68 and thermocouples 71 to 73. The tip of each thermocouple 71 to 73 is firmly connected to evaporation coil assemblies 43 to 45. The thermocouple 71 is located near the exit of duct 46 and are used to measure ambient temperature inside the evaporation assembly 29. Adjacent the top of each evaporation coil assemblies 43 to 45 are injectors 66 to 68, which are firmly attached to an adjacent evaporation platform 69C and have direct contact with the evaporation coil assemblies 43 to 45. The platforms 69C give the appearance of a spiral, which runs in a downward motion on each coil. Each injector is connected with stainless tubing to two separate bulkhead fittings. One bulkhead fitting delivers the chemical solution and the other the water separately to each injector. The outside part of each bulkhead is connected to capillary tubing consisting of 26 to 28 and 37 to 39. One set consisting of three tubes is connected to solenoids 21 to 23 and other set consisting also of three tubes (37 to 39) is connected to water manifold 36.

The air intake 42 into the evaporation assembly 29 is produced with fans 40 to 41 and the availability of a flow of air allows instant delivery of the transitional/molecular vapours from the evaporation assembly 29 via duct 46 into wet well 47.

Fan 40 runs continuously to perform dual functions. On the one hand it assists with the delivery of the vapours produced during the evaporation of the chemical agents (Group A-C) into wet well 47. On the other hand, it maintains a permanent air seal to prevent corrosive and contaminated air from entering of the wet well 47 into the evaporation assembly 29. The working mechanism of this air seal is simple. Fan 40 sucks air through duct 42 into the evaporation assembly 29 and once air is inside the evaporation assembly 29, the same fan pushes the air via duct 46 into the wet well 47. The pressure of air in duct 46 is higher than the air pressure in wet well 47. Thus the contaminated air stream in wet well 47 is denied entry into the evaporation system 29.

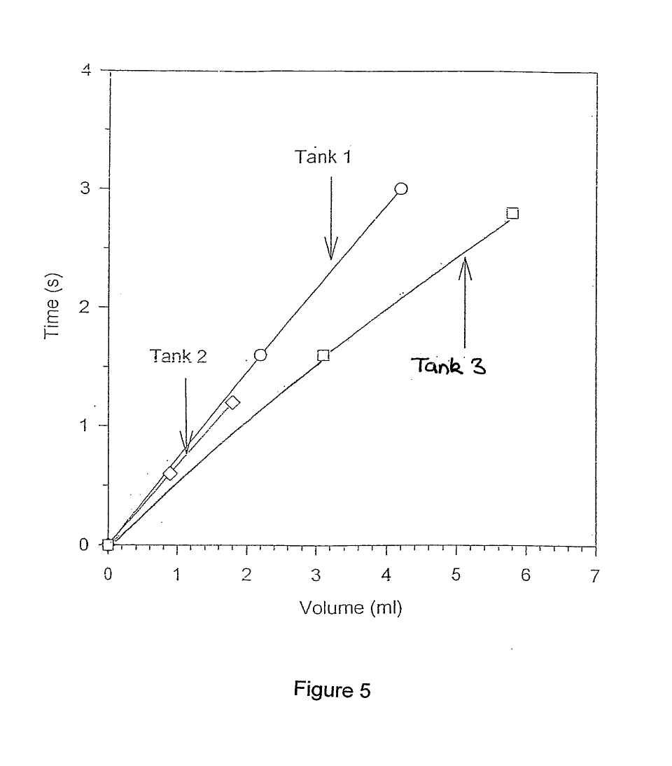

FIG. 5 shows low and high dosing rates, which relate to a precise volume of solutions, or water, being delivered to injectors 66 to 68. The dosing rate is a function of several variables which are as follows i.e. the time the solenoids 21 to 23 remain open, the aperture of the solenoids, the volume delivered per unit time, tank pressure, STP, specific gravity of solutions, size and length of capillary tubing 26 to 28 which are connected to injectors 66 to 68. The delivery rate of water into injectors 66 to 68 is also a function of the pressure of water in supply line 32, the size of the aperture of solenoid 34, the time the solenoid 34 remains open and the size and length of capillary tubing 37 to 39 connected to injectors 66 to 68.

As shown in FIG. 5, there are two main distinctions in the method used for dosing rates, (light and heavy). Light dosing means that a light chemical treatment is administered that requires light dosing. Light dosing and heavy dosing are partly interconnected and the differentiation is demonstrated in a practical example. Before highlighting this difference, the term chemical treatment versus no chemical treatment needs to be explained at the same time. Heavy treatment means separately injecting 3.2 ml of deodorising solution (Group A), 1.8 ml of detoxifying solution (Group B) and 3.6 ml of oxidising/disinfecting solution (Group C) onto each evaporation platform 69C and onto evaporation coils 63 to 65. Each platform 69C and individual coil is designated for each separate Group (A-C) of chemical agents. This regime of chemical treatment is repeated 8 times per hour (for a light treatment and 12 times per hour for a heavy treatment) unless the hydrogen sulphide or metrological override the preset values of the threshold functions, or the resting cycle has been changed. No treatment means that no further treatment is required. This status is determined by threshold settings and the analytes, which are measured at regular intervals.

FIG. 3 shows another important part of performing real time measurements of the concentration of hydrogen sulphide in vent 49A. Pipe 50A is an interconnection between connectors 48B and 51A. Connector 48B is jointed to vent pipe 49A and connector 51A is jointed to T-junction 52A. The top end of T-junction 52A is connected to the entry port 54 of air gate 56. The horizontal port 53A of T-junction 52A is connected to bleed pipe 53 through connector 53B. The electrical motor 55 is the direct drive for air gate 56 and facilitates opening and closing the same. In relation to operation of the real time H₂S sensor the PLC 1 controls the motor of air gate 56, air pump 58 and solenoids 59 and 60. When air gate 56 is open, gases from vent pipe 49A then stream through air gate 56 and these gases eventually accumulate in hydrogen sulphide sensor 62. When solenoid 60 and air gate 56 are open, the gases stream onto the hydrogen sulphide sensor 62 and consequently small amounts of the gaseous waste stream continuously escape through bleed pipe 61. This arrangement provides an open loop to prevent air locks and also accelerates stabilisation of the equilibrium of the gases in the H₂S sensor 62. After 20 seconds of signal integration time, the concentration value of the hydrogen sulphide is sent to PLC 1. Upon receipt of a signal, PLC 1 energises electrical motor 55 and closes air gate 56. Upon closure of air gate 56, air pump 58 and solenoid 59 are switched on and motor 55 is switched off. This purging process of hydrogen sensor 62, adaptor 57 and air gate 56 continues for 5 minutes but after 3 to 4 minutes, the digital readout on the H₂S sensor displays a reading 0.00 ppm.

The timing for purging of sensor 62 is largely determined by the concentration of the hydrogen sulphide that entered into the sensor compartment. After 5 minutes, air pump 58 and solenoids 59 to 60 are switched off and the hydrogen sulphide sensor is cleaned and sealed away until the next measurement is due. Solenoid 60 is not absolutely necessary if the hydrogen sulphide is installed in a hydrogen sulphide free environment.

Another feature of the air cleaning apparatus described herein is the independent time setting when the concentration of hydrogen sulphide in vent pipes 49A or in wet well 47 needs to be measured. The setting of sampling times, the time intervals for measuring the concentration of hydrogen sulphide in the gaseous waste stream are highly flexible and can be determined by the operator. In addition to these time and measurement regimes, the hydrogen sulphide threshold setting is another highly flexible feature of this disclosure.

The threshold setting of hydrogen sulphide refers to a differentiation between “low and high” concentrations of hydrogen sulphide escaping through vent pipe 49A and subsequently which treatment should be administered.

Preferably the air cleaning apparatus 10A of the disclosure is set to recognise a threshold of concentration of 5 ppm of hydrogen sulphide for low and a concentration of 10 ppm of hydrogen sulphide for high. Thus, a low threshold of hydrogen sulphide means, that, if the value is set for 18 ppm of hydrogen sulphide then, if the measured concentration of hydrogen sulphide is below 10 ppm, then a light chemical treatment will be administered. However, if the measured concentration of hydrogen sulphide is below the set threshold of 5 ppm, no chemical treatment will be administered. However, if the measured concentration of hydrogen sulphide is above the high set threshold value (10 ppm of hydrogen sulphide) a heavy treatment will be administered.

Another embodiment of the disclosure is the Override Threshold Function of the weather data with the H₂S concentration data. PLC 1 is connected via RS 232 cable to a weather station display panel (not shown). PLC 1 is programmed to collect data from the wireless weather station which sends wind velocity, wind span, wind direction, rain rate and barometric pressure data to PLC 1 every 30 seconds. PLC 1, in turn, is programmed to interrogate the data and activate the Override Threshold Function. In practical terms, that means that PLC 1 is capable of analysing the concentration of hydrogen sulphide combined with metrological data and, whenever necessary, will activate the Override Threshold Functions. The Override Threshold Function works as explained in the following example: Let us assume that the threshold for Wind Velocity is (2.0 m/s), for Wind Direction the setting is (centre 180°), for Wind Direction Span the threshold setting is (80°), for Rain Rate (the threshold is set for 2 mm/hr) and for Barometric Pressure (the threshold is set at 1002 hPa). Because the wind velocity threshold value is set at 2.0 m/s and wind velocity is above 2.0 m/s then, whether a light or heavy chemical treatment was determined using the H₂S data to be performed, will be cancelled. However, if the wind velocity is below the set threshold (2.0 m/s) then the chemical treatment will continue. Another example: The wind direction is reading 348°, the wind centre is set to 350° and the wind span is set between 310° to 360° (NW to N) and 0° to 30° (N to NE). Let us assume that a high-density population centre is located in the NE part of the pumping station and that the pumping station is in close proximity to the population centre. If the direction of the wind is coming from the NE and has a velocity above 2.0 m/s, then the chemical treatment will be cancelled. However, if the wind is blowing from the opposite direction 146° to 239° (SSE to SW) and if the wind velocity is above 2.0 m/s, then one of the chemical treatments will still be performed. The treatment to be administered will largely depend on the last reading of concentration of hydrogen sulphide. Regardless of the measured values of wind velocity and wind direction, if the measured rain rate is above 2 mm/hr, then the chemical treatment will be cancelled. Most odorous compounds escaping through the vent pipe 49A are water-soluble and will dissolve while it is raining.

A suitable setting of the threshold for barometric pressure is 1002 hPa. If the barometric pressure falls below this threshold but at the same time, the hydrogen sulphide threshold including wind velocity, wind direction and rain rate threshold values suggest that no chemical treatment is required, then all existing instructions will be cancelled and a heavy chemical treatment will be administered at once.

Another embodiment of this disclosure is the evaporation/heating, cooling and resting cycles. Evaporation/heating means heating up the coil assemblies 43 to 45 to approximately 162° C. to evaporate the chemical solutions and water on evaporation platforms 69C and on the coils 43 to 45. Cooling means to cool the coil assemblies with fans 40 to 41 and, if an accelerated cooling is required, solenoid 34 is activated during the cooling cycle, which permits the introduction of water via capillaries 37 to 39 to injectors 66 to 68. The introduction of water while the injectors are still hot also cleans the evaporation platforms 69C on the evaporation coil assemblies 43 to 45. Resting cycle means that the cooling cycle is completed and the resting cycle may run for 125 seconds until the PLC 1 determines the next operational step. If the treatment frequency needs to be increased, the time of the resting cycle must also be decreased.

Another innovative embodiment of this device is to perform a pyrolysis to facilitate cleaning of each evaporation platform 69C and evaporations coil assemblies 43 to 45. This treatment is performed using potent liquid cleaning agents, which are delivered via capillary tubing 26 to 28 while the evaporation coils are heated for 35 seconds.

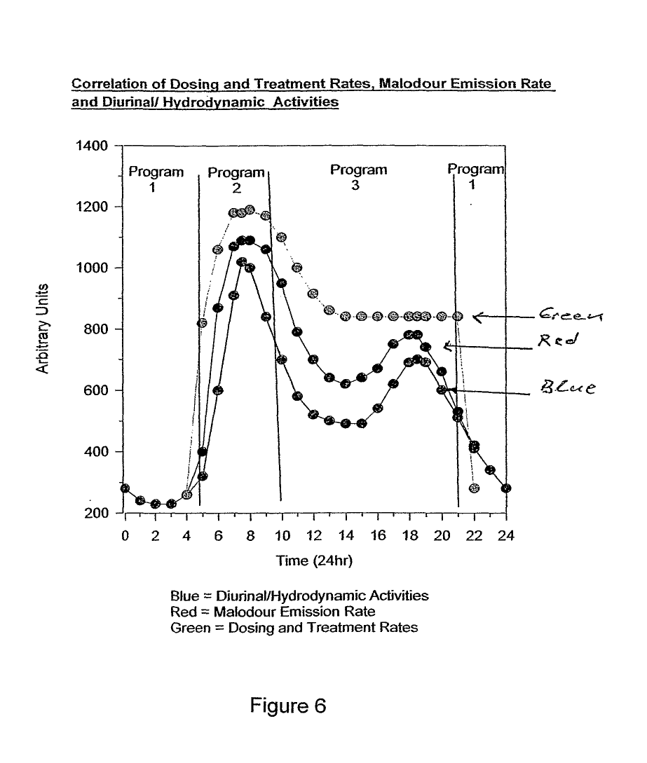

Deployment of threshold settings varies considerably among different operating wet wells to which parameters of the air cleaning apparatus is applied. For example, using the electronic controller which has been suitably programmed requires access to variables which are as follows:
1. Gaseous concentration of H₂S in wet well (high and low values) combined with the time of the operating cycle (see FIG. 6, program 1 to 3) over a 24 hour operating period.
2. Integration of volume of injection onto each evaporation platform based on concentration of H₂S measured over a 24 hour operating cycle.
3. Heating rates/heating time and final temperature of evaporation platform is also an important ingredient in the development of an effective and reliable algorithm.

A broad overview of these interactive operation processes is illustrated in FIG. 6 and highlights an elevated concentration of H₂S between a time of 0630 to 0930 in the morning which constitutes frequent treatments and heavy dosing rates. After a time of 0930 in the morning, the concentration of H₂S declines and lighter treatment and dosing rates are required. After a time of 2200 in the evening until 0630 the following morning no treatments are required. It must be noted that the patterns of odour/H₂S appearance and disappearance at different sewage pumping stations are not uniform being a function of many influences. Therefore an abatement of offensive odours and toxic gases is often at a most challenging task at certain sewage pumping stations.

There are considerable differences in comparing operating systems which can be selected to run the air cleaning apparatus of the disclosure. These systems include the following:

a) using an electronic controller which has been suitably programmed;

b) using a PLC with a real time operating system combined with relevant data input sensors; and

c) deployment of an array of integrated chips that are pre-programmed to replace an electronic controller or a real time PLC with sensor(s).

Electronic controllers, PLC's with sensor(s) data inputs and integrated chips are commercially available but with the skill of the programming these devices with accurate threshold values (see Table 2).

The disclosure in another aspect provides a process of decontamination of noxious or toxic odours from a wet well or other confined air space which includes the steps of:
(i) detecting the presence of said noxious or toxic odours in said wet well or other air space;
(ii) causing delivery of one or more decontamination solutions to an evaporation housing in fluid communication with the wet well or other confined air space wherein said one or more decontamination solutions come into contact with one or more associated coil assemblies which cause a phase change of said one or more decontamination solutions to a molecular activated species which
(iii) is then caused to flow into the wet well or confined air space to decontaminate same of said noxious or toxic odours.

An advantage of the air cleaning apparatus described above with reference to the drawings is that it enables an odourous air mass to be treated with active components in a way that delivers the active components very effectively to the air mass to be treated. This in turn leads to a reduced consumption of the active components to treat the air mass.

Yet further the air cleaning apparatus described above with reference to the drawings is able to deliver a level of treatment at a given time to match the demand that is required to treat the air mass at that particular time. Again this leads to a more effective use of the active components.

A further advantage of the air cleaning apparatus described above with reference to the drawings is that it is able to treat an odourous air mass more effectively than has previously been possible so as to substantially reduce odours issuing from a sewage well or sewage pump station. This is particularly important in built up areas where residential houses are built in close proximity to pump stations and wells and where the amenity of a region is reduced by malodour generated in waste water pump stations and wells.

A further advantage of the air treatment apparatus described above with reference to the drawings is that it can be applied across a wide range of applications. For example it can be applied in any situation where a body of air is generated malodourous compounds or harmful compounds that are escaping into the surrounding environment where they are having a deleterious effect.

It will of course be realized that the above has been given only by way of illustrative example of the disclosure and that all such modifications and variations thereto, as would be apparent to persons skilled in the art, are deemed to fall within the broad scope and ambit of the disclosure as is herein set forth.

TABLE 1

| Symbols | Thermocouples | Markers | Computation |
|---|---|---|---|
| M1 | (TC1, TC2, TC3) TC4 | M1 H | M1L ≧ |
| | | | (TC1, TC2, TC3) ≦ |
| | | | M1H |
| | | M1 L | M1L ≦ TC4 ≦ M1H |
| M2 | TC1, TC2, TC3 | M2 H | x̅ (TC1, TC2, TC3) ≦ |
| | | | M2H |
| | | M2 HH | x̅ (TC1, TC2, TC3) ≦ |
| | | | M2HH |
| M3 | TC1, TC2, TC3 | M3 | x̅ (TC1, TC2, TC3) ≦ M3 |
| M4 | TC1, TC2, TC3, TC4 | M4 | TC4 − |
| | | | M4 ≦ |
| | | | x̅ (TC1, TC2, TC3) ≦ |
| | | | TC4 + M4 |

Legend
TC1 means thermocouple 71
TC2 means thermocouple 72
TC3 means thermocouple 73
TC4 means thermocouple 74.

TABLE 2
Threshold settings

| | |
|---|---|
| Concentration of H₂S | (low 5 p.p.m) |
| Concentration of H₂S | (high 10 p.p.m) |
| Wind velocity | (2 m/s) |
| Wind direction | (348°) |
| Wind center | (350°) |
| Wind span | (80°) |
| Rain rate | (2 mm/hr) |
| Barometric pressure | (1002 hP) |
| Minimum volume in each tank | (0.5 L) |
| Setting of injector 1 | 1.6 ml (low), 3.2 ml (high) |
| Setting of injector 2 | 0.8 ml (low), 1.6 ml (high) |
| Setting of injector 3 | 1.8 ml (low), 3.6 ml (high) |
| Evaporation temperature | [M1 (H 60° C., L 25° C.)] |
| Evaporation temperature | [M2 (H 135° C., HH 150° C.)] |
| Cooling temperature | [M3 (148° C.)] |
| Cooling temperature | [M4 (55° C.)] |
| Heating rate | (7°/s) |
| Final temperature | (162° C.) |
| Evaporation time | (25 s) |
| Cooling time | (250 s) |
| Resting time | (125 s) |
| Optimal treatments | (12/Hr) |
| Minimal treatments | (6/Hr) |

What is claimed is:

1. Air cleaning apparatus for cleaning of air containing noxious or toxic odours which includes:
a controller for receiving real-time data from at least one sensor to control the operation of the air cleaning apparatus;
(ii) at least one tank having a decontamination solution;
(iii) a source of compressed air in fluid communication with the said at least one tank; and
(iv) an evaporation housing having at least one coil assembly in flow communication with the said at least one tank wherein said evaporation housing has an air inlet and an air outlet wherein the air outlet in use is in fluid communication with a wet well source of noxious or toxic odours whereby during operation of the air cleaning apparatus said decontamination solution is delivered under pressure to the said at least one coil assembly under the influence of the controller wherein a phase change of said decontamination solution occurs into an activated molecular species which is transferred into the wet well by air passing through the air inlet and the air outlet to reduce or eliminate said noxious or toxic odours from the wet well.

2. Air cleaning apparatus as claimed in claim 1 wherein the controller is an electronic controller.

3. Air cleaning apparatus as claimed in claim 1 which has at least three tanks containing decontamination solutions wherein a first tank contains a deodorising solution, a second tank contains a detoxifying solution and the third tank contains an oxidising/disinfecting solution and there are also provided at least three coil assemblies in the evaporation housing wherein a first coil assembly is in fluid communication with the first tank, a second coil assembly is in fluid communication with the second tank and a third coil assembly is in fluid communication with the third tank.

4. Air cleaning apparatus as claimed in claim 3 wherein the first tank contains one or more solutions selected from ethanol aldehydes, ketones, chloroxylenol, benzalkonium chloride, essential oils, cyclodextrins, isopropyl alcohol, orris concrete, oleoresins, orris root, chlorohexidine and esters.

5. Air cleaning apparatus as claimed in claim 3 wherein the second tank contains one or more solutions selected from di-isopropyl amine, diglycolamine, monoethanolamine, pyrimethamine, acetonitrile, vinorelbine ditartrate, methonal, xylitol n-hydroxyethyl piperidine, ammonium chloride, ferric chloride, ferric hydroxide, caustic soda and hydrogen peroxide.

6. Air cleaning apparatus as claimed in claim 3 wherein the third tank contains one or more solutions selected from ethanol, isopropyl alcohol, water, acetic acid and hydrogen peroxide.

7. Air cleaning apparatus as claimed in claim 1 wherein there is provided a single flow conduit between a single tank and a single coil assembly having a flow control valve and a solenoid.

8. Air cleaning apparatus as claimed in claim 3 wherein there is provided a first flow conduit between the first tank and the first coil assembly, a second flow conduit between the second tank and the second coil assembly and a third flow conduit between the third tank and the third coil assembly wherein each of the first, second and third flow conduits also have a flow control valve and a solenoid.

9. Air cleaning apparatus as claimed in claim 1 wherein the evaporation housing includes one or more fans or air blowers to provide a forced draught of air between the inlet and the outlet.

10. Air cleaning apparatus as claimed in claim 1 wherein there is provided a mechanism responsive to pressure such as a high pressure relief valve or a mechanism including a pressure sensor to maintain air pressure in said tank(s) containing decontamination solution below a predetermined operating pressure.

11. Air cleaning apparatus as claimed in claim 1 wherein there is provided a liquid/gas separator in flow communication with said tank(s) containing decontamination solution to remove tiny air bubbles when hydrogen peroxide is used as a decontamination solution.

12. Air cleaning apparatus as claimed in claim 1 wherein there is provided a water conduit in flow communication with injectors located as part of the or each coil assembly wherein water is discharged onto an adjacent coil assembly to cool the adjacent coil assembly when required.

13. Air cleaning apparatus as claimed in claim 12 wherein said water conduit also contains a flow control valve and a solenoid.

14. Air cleaning apparatus as claimed in claim 1 wherein the or each coil assembly is provided with thermocouples at each end thereof to facilitate monitoring of heating/cooling profiles of the or each coil assembly.

15. Air cleaning apparatus as claimed in claim 1 wherein the or each coil assembly includes a coil wound or wrapped around a solid coil support to provide an evaporation platform of relatively large surface area to facilitate production of said activated molecular species.

16. Air cleaning apparatus as claimed in claim 1 wherein there is provided a vent pipe extending from said wet well which contains said noxious or toxic odours and said vent pipe in use is in fluid communication with an air gate of said air cleaning apparatus which is driven by a drive motor for opening and closing of same and said air gate when open is in fluid communication with a sensor for detection of said noxious or toxic odours when activated by the controller causes delivery of decontamination solution(s) to associated coil assembly(s) when required to purge said noxious or toxic odours from the wet well.

17. Air cleaning apparatus as claimed in claim 16 wherein the air gate is provided with a solenoid as well as an air pump for purging of noxious or toxic odours from the sensor/s.

18. Air cleaning apparatus as claimed in claim 1 wherein said controller is a PLC having a data input connected to one or more real time sensors and a data output connected to said at least one tank, said source of compressed air and said evaporation housing.

19. Air cleaning apparatus as claimed in claim 1 wherein said electronic controller is one or more integrated chips connected to one or more real time sensors and also connected to said at least one tank, said source of compressed air and said evaporation housing.

20. Air cleaning apparatus as claimed in claim 19 wherein the data output is also connected to one or more injectors for injecting decontamination solution into the evaporation housing.

21. Air cleaning apparatus as claimed in claim 1 wherein the controller includes one or more of a programmable logic controller and an integrated chip.

22. A process of decontamination of noxious or toxic odours from a wet well which includes the steps of:
(i) detecting a real-time presence of said noxious or toxic odours in said wet well;
(ii) causing delivery of one or more decontamination solutions to an evaporation housing in fluid communication with the wet well wherein said one or more decontamination solutions come into contact with one or more associated coil assemblies which cause a phase change of said one or more decontamination solutions to a molecular activated species; and
(iii) causing said decontamination solution(s) to flow into the wet well to decontaminate same of said noxious or toxic odours.

23. A process as claimed in claim 22 wherein the molecular activated species is caused to move into the wet well or air space by a forced draught of air passing through the evaporation housing.

24. A process as claimed in claim 22 wherein the or each coil assembly has a coil wound around a solid support to provide an area of relatively large surface area or evaporation platform to facilitate production of said molecular activated species.
